(19) United States
(12) Patent Application Publication
(10) Pub. No.: US 2007/0016036 A1
(43) Pub. Date: Jan. 18, 2007

(54) Ultrasonic diagnostic apparatus and image processing method

(75) Inventor: Masahide Nishiura, Kanagawa (JP)

Correspondence Address: AMIN, TUROCY & CALVIN, LLP, 1900 EAST 9TH STREET, NATIONAL CITY CENTER, 24TH FLOOR,, CLEVELAND, OH, 44114, US

(73) Assignee: Kabushiki Kaisha Toshiba, Minato-ku (JP)

(21) Appl. No.: 11/438,584

(22) Filed: May 22, 2006

(30) Foreign Application Priority Data
May 23, 2005 (JP) 2005-150233

(51) Int. Cl. A61B 8/00

(52) U.S. Cl. 600/437

(57) ABSTRACT

An ultrasonic diagnostic apparatus can obtain an image having an improved S/N ratio irrespective of the existence of a movement of an object and the existence of periodicity of the movement, and includes a first image generation part to generate images from reflected signals from a moving object to be tested, a displacement detection part to detect a movement between the time-series images, a displacement correction part to generate, based on the detected movement, displacement corrected received signals in which displacement is corrected, a signal addition part to weight-add the plural displacement corrected received signals, and a second image generation part to convert an added received signal into an image for display.

### CROSS REFERENCE TO RELATED APPLICATIONS

This application is based upon and claims the benefit of priority from the prior Japanese Patent Application No. 2005-150233, filed on 23 May, 2005; the entire contents of which are incorporated herein by reference.

### BACKGROUND OF THE INVENTION

The present invention relates to an ultrasonic diagnostic apparatus, and particularly to an ultrasonic diagnostic apparatus having a function to reduce noise of an image generated by the ultrasonic diagnostic apparatus and an image processing method of the same.

In an ultrasonic diagnostic apparatus, an ultrasonic pulse wave is transmitted into a human body, an echo signal which is reflected due to the existence of a difference in acoustic impedance between living tissues is received, the intensity of the echo signal is imaged, and the tissue shape or the like in the human body can be seen.

The echo signal includes noises due to various causes, and when the amount of the noises is large, the diagnosis is hindered. Thus, it is an important problem to reduce the noise. That is, it is an important problem to improve a signal-to-noise ratio (S/N ratio).

Among various noises, with respect to a random noise having low auto-correlation, the S/N ratio can be improved by adding plural signals. This is based on the principle that when n signals are added, although a primary signal component (reflected signal from a living body) having high auto-correlation is increased by a factor of n, a random noise is merely increased by a factor of ⁻n as an expected value.

Accordingly, when signals of plural time phases are added from among time-series signals obtained by the ultrasonic diagnostic apparatus, the S/N ratio can be improved.

However, this method is not effective unless an object exists at the same position in the plural time phases. That is, in the case where the object moves, this method can not be used.

On the other hand, JP-A-10-118061 or JP-A-2001-170047 discloses a method in which with respect to a periodically moving object to be tested, plural image frames in which movements have the same phase are added.

When the noise of an image obtained by the ultrasonic diagnostic apparatus is large, the diagnosis is hindered. In order to improve the signal-to-noise ratio (S/N ratio), a method is conceivable in which plural signals are added, however, there is no effect in the case where an object moves.

Besides, in the method in which with respect to the periodically moving object to be tested, plural image frames having the same phase in the movement are added, it is necessary that the object is in periodic motion, and this method can not be used for a nonperiodically moving object. Further, a memory for holding image data in one period or more is needed, and image frames having the same phase need to be made to correspond to each other.

Then, the invention has been made to solve the problems, and has an object to provide an ultrasonic diagnostic apparatus which can obtain an image with an improved S/N ratio irrespective of the existence of movement of an object and the existence of periodicity of the movement, and a method of the same.

### BRIEF SUMMARY OF THE INVENTION

According to an aspect of the invention, an ultrasonic diagnostic apparatus drives an ultrasonic probe, configured to transmit/receive ultrasonic waves, to transmit ultrasonic waves to an object to be tested, receives the ultrasonic waves reflected from the object to be tested in time-series, converts time-series received signals thereof into time-series images and displays them, and the ultrasonic diagnostic apparatus includes a first image generation unit to convert the time-series received signals into time-series images for displacement detection, a displacement detection unit to detect information relating to displacement between the converted time-series images for displacement detection, a displacement correction unit which inversely converts the information relating to the displacement and generates, based on the inversely converted information relating to the displacement, plural displacement corrected signals in which a position shift due to a movement of the object to be tested is corrected, an addition unit to generate an added received signal by weight-adding the plural displacement corrected received signals, and a second image generation unit to convert the added received signal into an image for display.

According to another aspect of the invention, an ultrasonic diagnostic apparatus drives an ultrasonic probe, configured to transmit/receive ultrasonic waves, to transmit ultrasonic waves to an object to be tested, receives the ultrasonic waves reflected from the object to be tested in time-series, converts time-series received signals thereof into time-series images and displays them, and the ultrasonic diagnostic apparatus includes an image generation unit to convert the time-series received signals into time-series images for displacement detection, a displacement detection unit to detect information relating to displacement between the time-series images for displacement detection, a displacement correction unit which generates, based on the information relating to the displacement, displacement corrected images in which a position shift due to a movement of the object to be tested is corrected, and an addition unit to generate an image for display by weight-adding the plural displacement corrected images.

According to another aspect of the invention, an ultrasonic diagnostic apparatus drives an ultrasonic probe, configured to transmit/receive ultrasonic waves, to transmit ultrasonic waves to an object to be tested, receives the ultrasonic waves reflected from the object to be tested in time-series, converts time-series received signals thereof into time-series images and displays them, and the ultrasonic diagnostic apparatus includes a displacement detection unit to detect information relating to displacement between the time-series received signals, a displacement correction unit which generates, based on the information relating to the displacement, plural displacement corrected received signals in which a position shift due to a movement of the object to be tested is corrected, an addition unit to generate an added received signal by weight-adding the plural displacement corrected received signals, and an image generation unit to convert the added received signal into an image for display.

According to another aspect of the invention, an ultrasonic diagnostic apparatus drives an ultrasonic probe, configured to transmit/receive ultrasonic waves, to transmit ultrasonic waves to an object to be tested, receives the ultrasonic waves reflected from the object to be tested in time-series, converts time-series received signals thereof into time-series images and displays them, and the ultrasonic diagnostic apparatus includes a displacement detection unit to detect information relating to displacement between the time-series received signals, a displacement correction unit which generates, based on the information relating to the displacement, plural displacement corrected received signals in which a position shift due to a movement of the object to be tested is corrected, an image conversion unit to convert the plural displacement corrected received signals into displacement corrected images, and an addition unit to generate an image for display by weight-adding the plural displacement corrected images.

According to the aspects of the invention, in the ultrasonic diagnostic apparatus, an image having an improved S/N ratio can be obtained irrespective of the existence of movement of an object, and the existence of periodicity of the movement.

### BRIEF DESCRIPTION OF THE DRAWINGS

FIG. 1 is a block diagram showing a structure of an ultrasonic diagnostic apparatus according to a first embodiment of the invention.

FIG. 2 is a view for explaining a procedure of generating image data from ultrasonic signals in the ultrasonic diagnostic apparatus.

FIG. 3 is a view exemplifying a displacement detection processing in a displacement detection part of the ultrasonic diagnostic apparatus according to the first embodiment.

FIG. 4 is a view showing a generation processing of a displacement corrected signal in a signal addition part of the ultrasonic diagnostic apparatus according to the first embodiment.

FIG. 5 is a view showing a frame data addition operation in the signal addition part of the ultrasonic diagnostic apparatus according to the first embodiment.

FIG. 6 is a block diagram showing a structure of an ultrasonic diagnostic apparatus according to a second embodiment.

FIG. 7 is a view showing a generation procedure of a displacement corrected image in a signal addition part of the ultrasonic diagnostic apparatus according to the second embodiment.

FIG. 8 is a view showing a frame image addition operation in the signal addition part of the ultrasonic diagnostic apparatus according to the second embodiment.

FIG. 9 is a block diagram showing a structure of an ultrasonic diagnostic apparatus according to a third embodiment.

FIG. 10 is a block diagram showing a structure of an ultrasonic diagnostic apparatus according to a fourth embodiment.

### DETAILED DESCRIPTION OF THE INVENTION

Hereinafter, embodiments of the invention will be described with reference to the drawings.

### First Embodiment

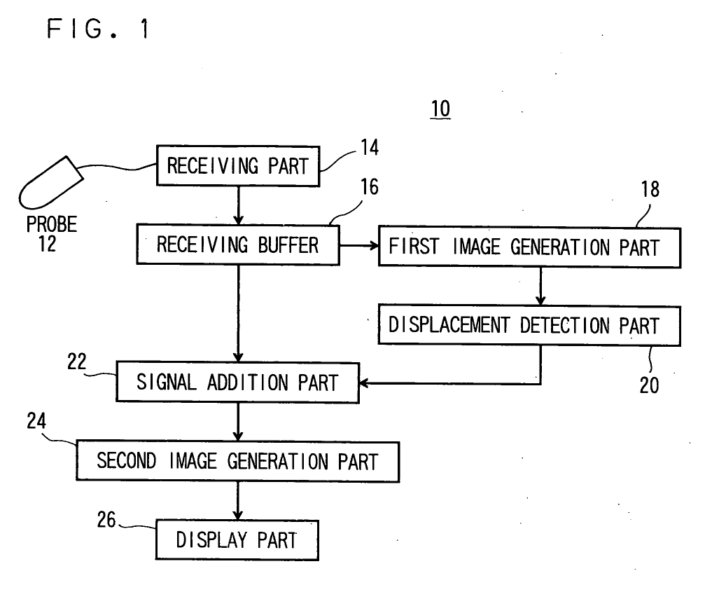

An ultrasonic diagnostic apparatus 10 according to a first embodiment of the invention will be described with reference to FIGS. 1 to 5.

### (1) Structure of Ultrasonic Diagnostic Apparatus 10

FIG. 1 is a block diagram showing the ultrasonic diagnostic apparatus 10 of this embodiment.

The ultrasonic diagnostic apparatus 10 includes an ultrasonic probe 12 configured to transmit/receive ultrasonic waves, a receiving part 14 which drives the ultrasonic probe 12 to transmit ultrasonic waves, receives reflected ultrasonic waves, and performs an A/D conversion processing to convert them into received signals, a receiving buffer 16 to hold the converted received signals, a first image generation part 18 which performs a filter operation or a scan conversion operation on the received signals and generates image data, a displacement detection part 20 to detect from the image data the displacement between frames at each local part of an object to be tested, a signal addition part 22 to weight-adding received signals of plural frames by using the detected displacement information, a second image generation part 24 to generate image data for display by performing a filter operation or a scan conversion operation on the added received signal, and a display part 26 to display the image data for display.

### (2) Receiving Part 14 and Receiving Buffer 16

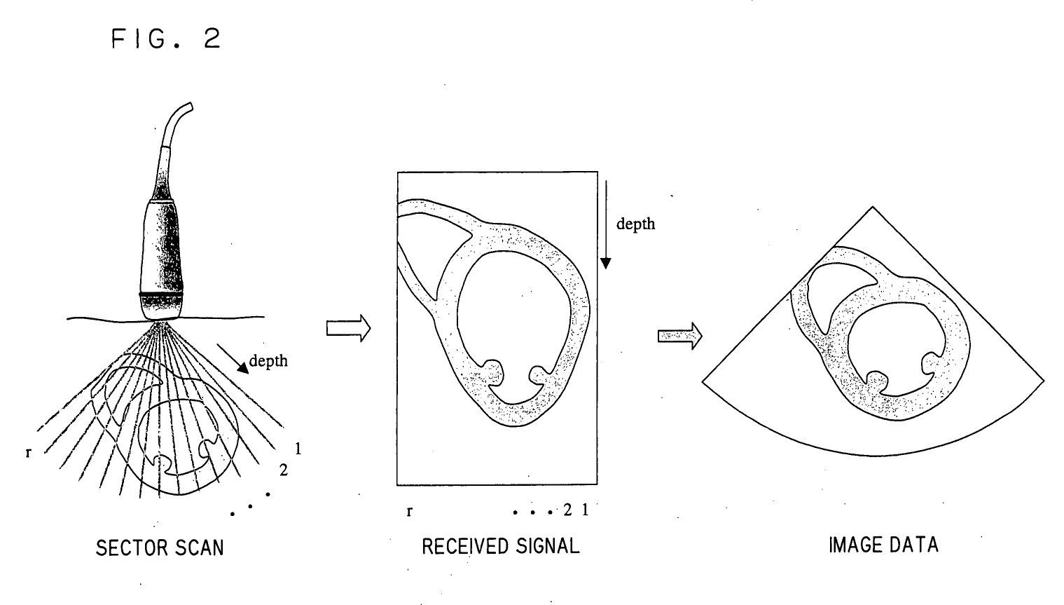

An ultrasonic beam is emitted from the ultrasonic probe 12 driven by the receiving part 14. The ultrasonic waves reflected by the moving object to be tested are received by the ultrasonic probe 12 and are sector scanned (see the left of FIG. 2), and are converted into time-series received signals in the receiving part 14 by detection and A/D conversion processing (see the middle of FIG. 2). The time-series received signals are a list of data in each ultrasonic beam direction, and data of one frame are formed of data in a series of beam directions. The receiving buffer 16 holds time-series received signals over plural frames.

### (3) First Image Generation Part 18

The time-series received signals held by the receiving buffer 16 are subjected to a filter operation or a scan conversion operation by the first image generation part 18 and becomes time-series image data for displacement detection (see FIG. 2). In the first image generation part 18, it is appropriate that parameters of the filter operation or the scan conversion operation are set so that image characteristics of the image data to be generated become suitable for displacement detection at a later stage. The generated image data are stored in an image buffer provided in the first image generation part 18. This image buffer has such capacity as to hold the image data for displacement detection in plural frames.

### (4) Displacement Detection Part 20

Next, the displacement detection part 20 uses the time-series image data for displacement detection held in the image buffer and detects the displacement of each local part of the moving object to be tested between the temporally adjacent frames. The displacement detection can be performed by, for example, block matching at each local part.

Although the displacement may be detected at all positions, a structure may be made such that the displacement is detected at discrete positions according to objective resolution, and the displacement at all positions can be calculated by interpolation.

The movement of each local part of the object to be tested between the frames of the images for displacement detection becomes clear by the displacement detection processing, and even if the object to be tested is moved or deformed, the corresponding position of each local part becomes understandable.

As the displacement detection processing, in addition to the method by the block matching, another method can also be used.

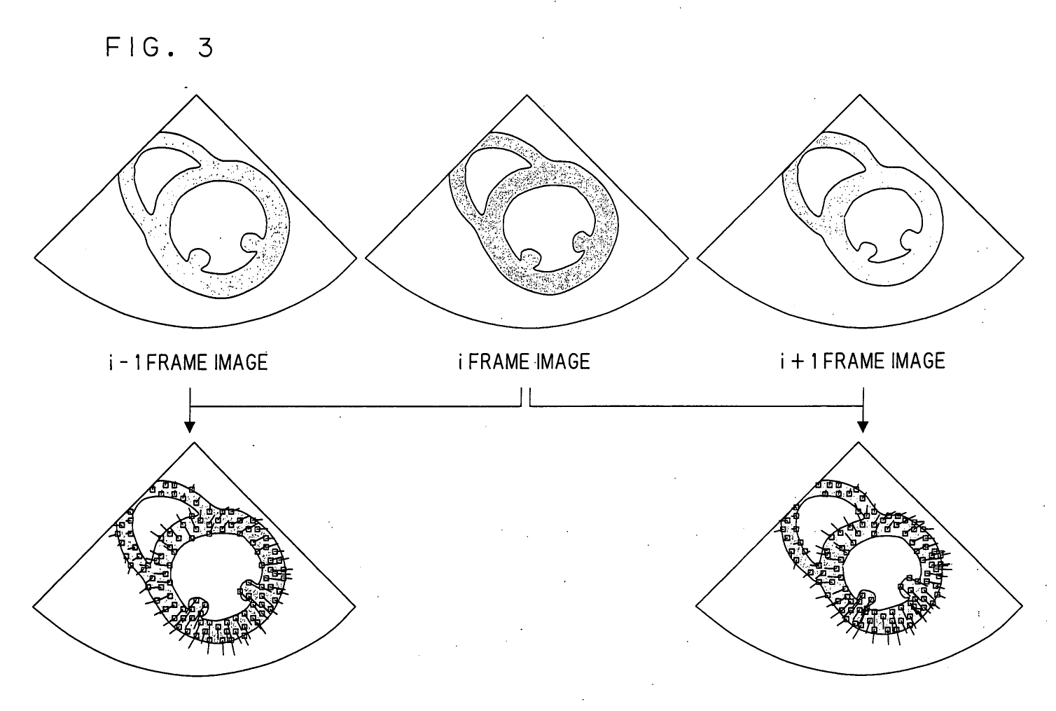

FIG. 3 shows an example of displacement between frames of images for displacement detection. There is shown the example of the displacement detection of the object to be tested between temporally continuous (i−1)th frame image and ith frame image, and between the ith frame image and (i+1)th frame image. Since the difficulty of the displacement detection varies according to the shape of the object to be tested and the properties of the image such as the existence of texture, when the displacement at a position where the displacement is easy to detect is obtained, efficiency is excellent in view of accuracy and processing speed.

FIG. 3 schematically illustrates a state in which displacement of each local portion of an object to be tested (heart in the example of this drawing) is detected at respective feature point. Each square in the drawing indicates a position where the displacement is detected, and a line segment extending from the square indicates the direction and magnitude of the displacement. The each feature point indicates the position of an edge on the image where an edge processing on the image is performed. Incidentally, the feature point may indicate a position obtained by corner detection or peak detection on an image.

However, it is not always necessary that the position where the displacement detection is performed is the feature point, and it can be performed at all pixel positions.

### (5) Signal Addition Part 22

Next, a processing in the signal addition part 22 is performed. The processing of the signal addition part 22 is roughly divided into three parts, that is, a displacement inverse conversion processing, a displacement corrected received signal generation processing, and a signal addition processing.

(5-1) First Processing

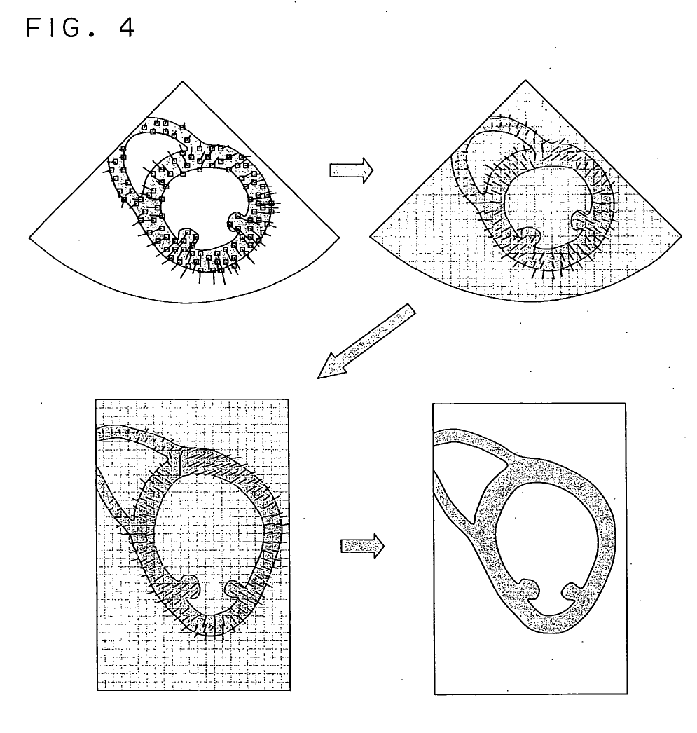

As the first processing, displacement information between frames of images for displacement detection detected by the displacement detection part 20 is inversely converted into displacement in the coordinate system of the received signal before the scan conversion (see the upper left of FIG. 4).

In the processing method exemplified in FIG. 3, the displacement information detected by the displacement detection part 20 is obtained only on the feature point position. Then, the displacement information obtained on the feature point is interpolated spatially, so that the displacement at an arbitrary position can be calculated (see the upper right of FIG. 4). For example, in the case of a sectional image, two-dimensional interpolation is performed, and in the case of three-dimensional data, the displacement information becomes a three-dimensional vector, and the interpolation is performed three-dimensionally.

The displacement in the coordinate system of the received signal is generated by the reverse conversion of the scan conversion from the received signal to the image data (see the lower left of FIG. 4).

Incidentally, a structure can also be made such that the interpolation of the displacement information is not performed on the image data, and after the displacement information at the feature point position is converted into that in the received signal coordinate system, the interpolation is spatially performed on the received signal.

(5-2) Second Processing

As the second processing, a displacement corrected received signal in which displacement is corrected is generated using displacement in the coordinate system of the received signal (see the lower right of FIG. 4).

It is read from the displacement in the coordinate system of the received signal that a position (position A) of an ith frame was which position (position B) of an (i−1)th frame, and data at the position B of the received signal of the (i−1)th frame is made data at the position A of the displacement corrected received signal corresponding to the (i−1)th frame.

Similarly, it is read from the displacement in the coordinate system of the received signal that a position (position A) of the ith frame becomes which position (position B) of an (i+1)th frame, and data at the position B of the received signal of the (i+1)th frame is made data at the position A of the displacement corrected received signal corresponding to the (i+1)th frame.

In the case where the data position is not an integer, what is obtained by interpolating surrounding data may be used.

By generating the displacement corrected received signal as stated above, the displacement corrected received signals including the received signal data of the (i−1)th and (i+1)th frames corresponding to (shift due to the movement is corrected) the position of the object to be tested in the ith frame can be generated.

(5-3) Third Processing

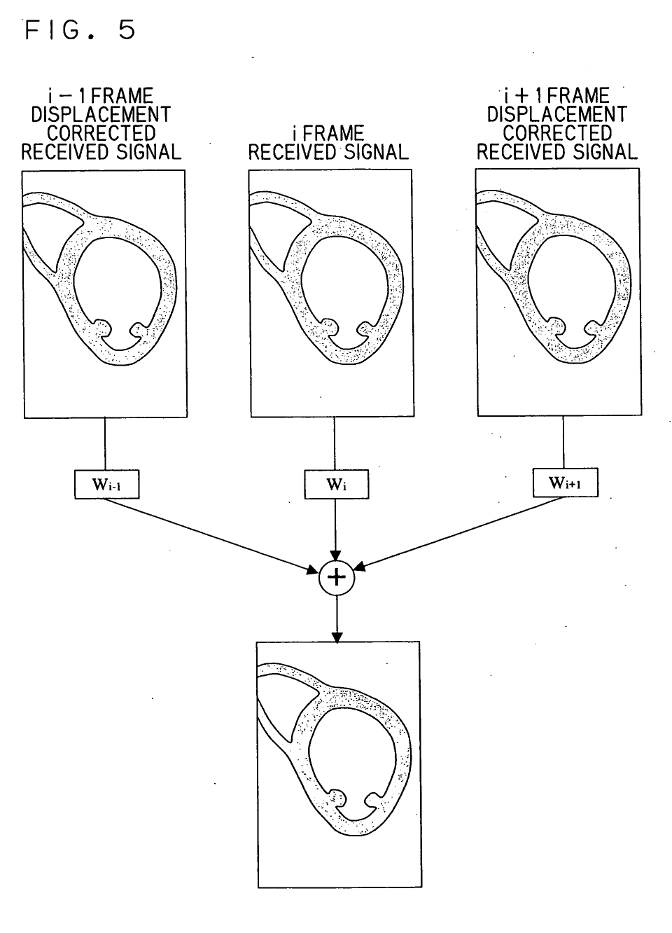

As the third processing, the displacement corrected received signals of plural frames are weight-added to generate an added received signal (see FIG. 5).

By the principle that when signals of n frames are added, although a primary signal component (reflected signal from a living body) having high auto-correlation is increased by a factor of n, random noise is merely increased by a factor of ⁻n as an expected value, the added received signal becomes a signal in which the S/N ratio is improved. Besides, since the movement of the object to be tested is corrected at the time of addition, even in the case where the object to be tested is moved or deformed, there is an effect.

Incidentally, FIGS. 3 and 5 exemplify the case of n=3. A structure can also be made such that the addition of more frames is performed. In that case, although displacement between frames separated from each other by two frames or more can be calculated by obtaining displacement between continuous frames and by accumulating the displacement, the displacement may be directly detected between a noted frame and an objective frame.

With respect to a weight coefficient w at the time of addition, for example, when it is 1/n, an average value of the respective frames is taken. Alternatively, it is also possible to change the weight coefficient by the accuracy of a displacement detection result. For example, in the case where the detection accuracy of the displacement is excellent, the weight coefficients of the respective frames are made uniform, and in the case where the accuracy is poor, the weight of a noted frame (the ith frame in the example) is made larger than those of the other frames. By doing so, a bad influence, such as a blur of an added image, occurring in the case where the detection accuracy of the displacement is poor, can be reduced.

Similarly, the number n of the frames used for the addition can also be dynamically changed according to the accuracy of displacement detection or the magnitude of displacement. For example, when the number n of the addition frames is made large in the case where the detection accuracy is good, and when the number n of the addition frames is made small in the case where the detection accuracy is poor, while a bad influence due to an error, such as a blur of an added image, is suppressed, the merit of the improvement of the S/N ratio can be effectively obtained. Alternatively, when the number of addition frames is made small/large according to large/small of the displacement respectively, a higher effect can be obtained.

Incidentally, when plural frames are added, there occurs a case where a correspondence position is outside the range of data according to the movement of the object to be tested. In this case, it is appropriate that the frame having no data with respect to the position is removed from objects of the addition, and the weight coefficient is adjusted.

(6) Second Image Generation Part 24 and Display Part 26

In the second image generation part 24, the added received signal is subjected to the filter operation or the scan conversion operation and image data for display is generated. The generated image data for display is presented to the user by the display part 26.

As stated above, according to this embodiment, also in the case where the object to be tested is moved or deformed, the image in which the S/N ratio is improved can be obtained. Besides, since the received signals are added, there is a merit that there is no influence of the filter operation or brightness conversion at the time of generation of an image for display. Further, while the image data is expressed in 256 gradations of 8 bits in many cases, the received signal is generally expressed by more bits (for example, 16 bits), and therefore, the image in which S/N is improved can be obtained by weight-adding the displacement corrected received signals to generate the added received signal.

### (7) Modified Example

The image data generated in the first image generation part 18 is the image data for displacement detection, which is used for displacement detection, it is not necessary to generate the image by the same parameters as those at the time when the image data for display is generated in the second image generation part 24. Setting can be made such that the resolution suitable for the displacement detection and the filter operation are made parameters.

### Second Embodiment

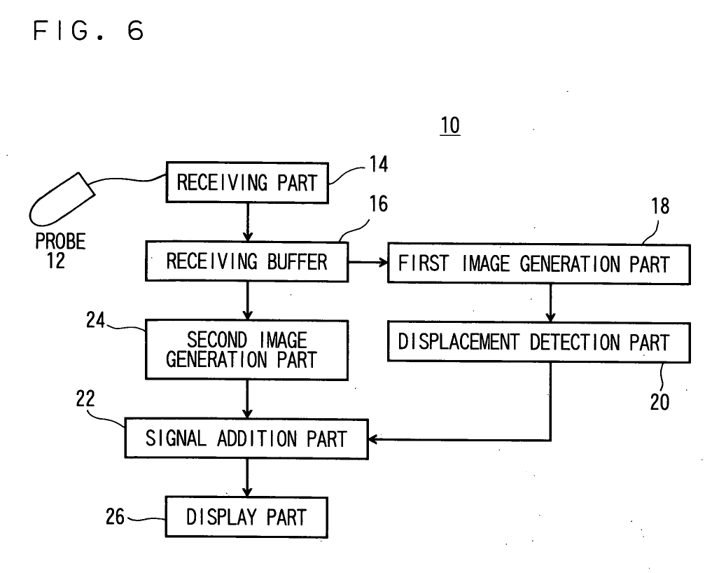

An ultrasonic diagnostic apparatus 10 according to a second embodiment of the invention will be described with reference to FIGS. 6 to 8. This embodiment is different from the first embodiment in that signal addition is performed on image data.

FIG. 6 is a block diagram showing the ultrasonic diagnostic apparatus 10 according to this embodiment.

The ultrasonic diagnostic apparatus 10 includes an ultrasonic probe 12 configured to transmit/receive ultrasonic waves, a receiving part 14 which drives the ultrasonic probe 12 to transmit ultrasonic waves, receives the reflected ultrasonic waves, and performs A/D conversion processing to convert them into received signals, a receiving buffer 16 to hold the converted received signals, a first image generation part 18 which performs a filter operation or a scan conversion operation on the received signals and generates image data for displacement detection, a displacement detection part 20 to detect displacement of image data between frames at each local part of an object to be tested, a second image generation part 24 to perform a filter operation or a scan conversion operation on the received signals to generate image data for display, a signal addition part 22 which uses detected displacement information and weight-adds the image data for display in plural frames, and a display part 26 to display the added image data for display.

The transmission/reception of the ultrasonic waves, the generation of image data for displacement detection from the received signals, and the detection portion of displacement are the same as the flow of the processing of the first embodiment.

In this embodiment, the data to be added in the signal addition part 22 is the image data for displacement detection. While the displacement detection processing is performed, an image for display is generated in the second image generation part 24. In the second image generation part 24, the image data for display is generated from the received signals held in the receiving buffer 16 by the filter operation or the scan conversion operation.

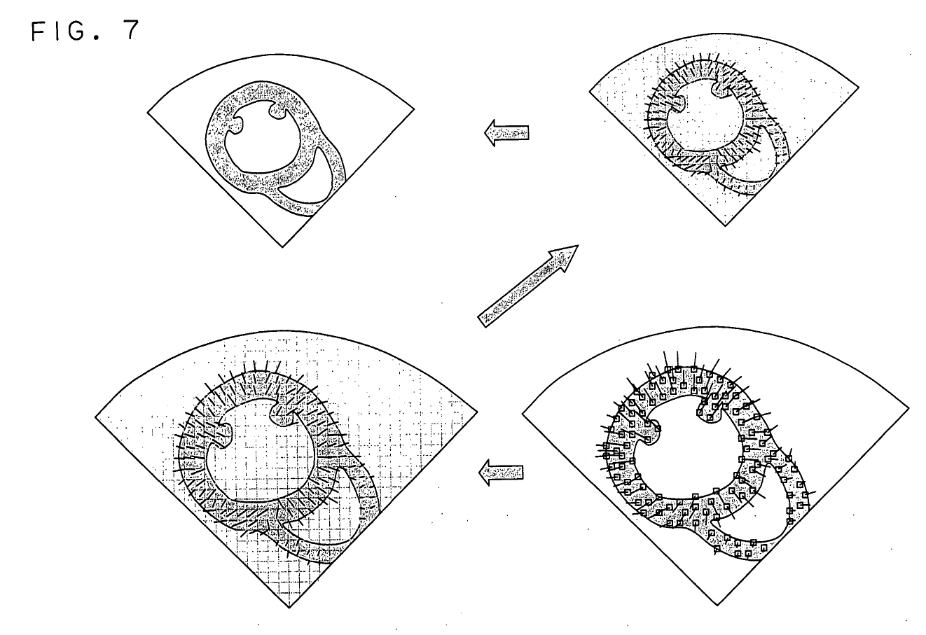

The signal addition part 22 first converts displacement information between frames of images for displacement detection detected in the displacement detection part 20 into displacement in the coordinate system of the frame of the image for display, and uses the converted displacement to obtain displacement corrected image data. FIG. 7 shows an example of conversion of displacement in the case where the resolution is different between the image data for displacement detection and the image data for display. Also in this embodiment, since the displacement information detected in the displacement detection part 20 relates to only the feature point positions, the displacement information obtained on the feature point is spatially interpolated, so that displacement at an arbitrary position can be calculated (see the upper right of FIG. 7). That is, in the case of a sectional image, two-dimensional interpolation is performed, and in the case of three-dimensional data, the displacement information also becomes a three-dimensional vector, and the interpolation is also performed three-dimensionally. Thereafter, the displacement information is converted into displacement information on the image data for display (see the lower left of FIG. 7), and a displacement corrected image is generated (see the lower right of FIG. 7).

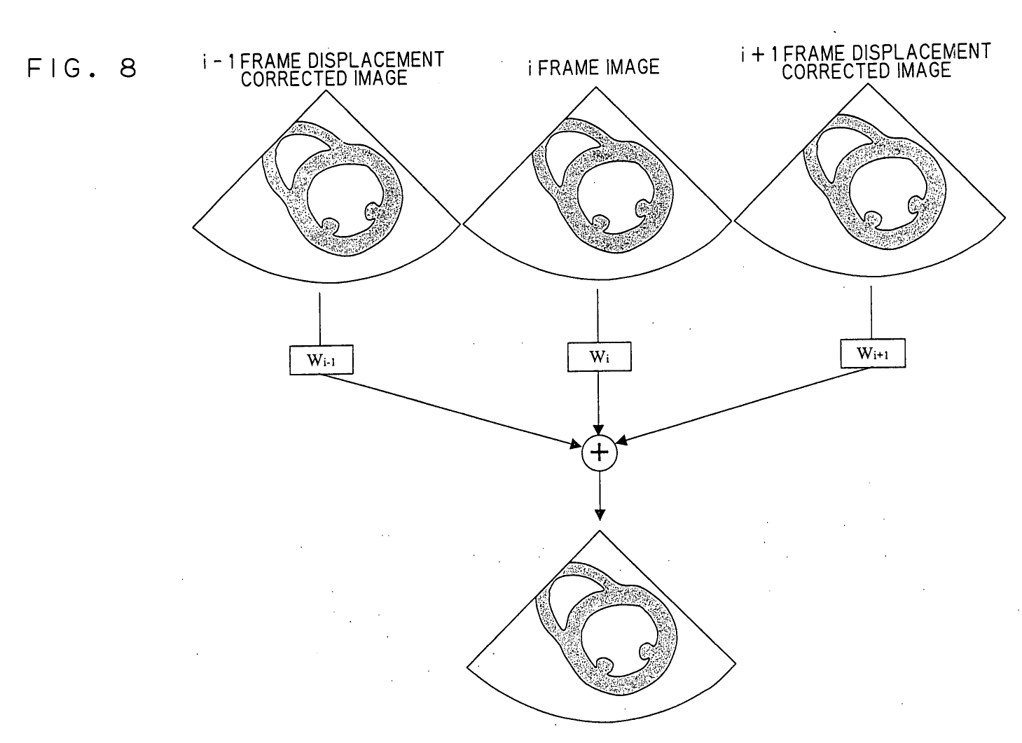

Further, in the signal addition part 22, displacement corrected images of plural frames are weight-added to generate an added image (see FIG. 8). Even in the case where addition is performed on the image data, by the principle that when signals of n frames are added, although a primary component (reflected signal from a living body) having high auto-correlation is increased by a factor of n, random noise is merely increased by a factor of n as an expected value, the added image becomes the image in which the S/N ratio is improved.

Finally, the added image is presented to the user by the display part 26.

When the structure is made such that the image data is added as in this embodiment, there is a merit that the coordinate system conversion of the displacement is completed by only the conversion of a scale.

Incidentally, in this embodiment, although the first image generation part 18 and the second image generation part 24 are separated from each other, a structure may be made such that these two image generation parts are made one common part, and image data for displacement detection and image data for display are made the same. By doing so, the structure of the apparatus can be simplified.

### Third Embodiment

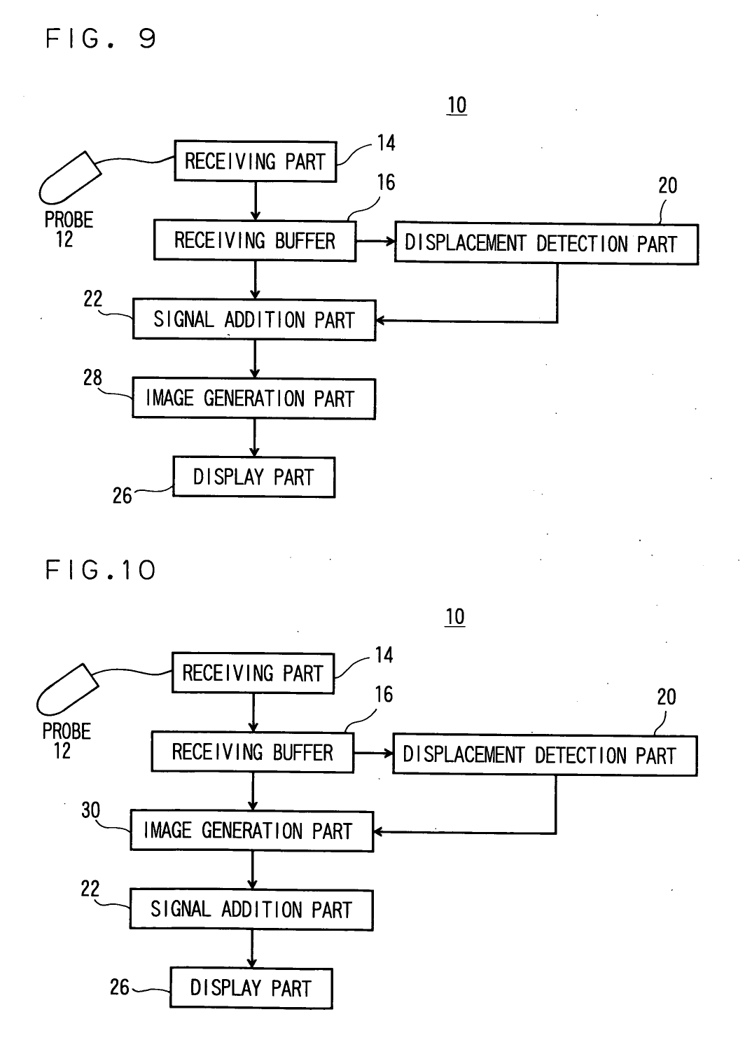

An ultrasonic diagnostic apparatus 10 according to a third embodiment of the invention will be described with reference to FIG. 9. This embodiment is different from the first embodiment in that displacement detection is performed on a received signal.

FIG. 9 is a block diagram showing the ultrasonic diagnostic apparatus 10 of this embodiment.

The ultrasonic diagnostic apparatus 10 includes an ultrasonic probe 12 configured to transmit/receive ultrasonic waves, a receiving part 14 which drives the ultrasonic probe 12 to transmit ultrasonic waves, receives the reflected ultrasonic waves, and performs A/D conversion to convert them into received signals, a receiving buffer 16 to hold the converted received signals, a displacement detection part 20 to detect displacement between frames of the received signals at each local part of an object to be tested, a signal addition part 22 to weight-add the received signals of plural frames by using detected displacement information, an image generation part 28 which performs a filter operation or a scan conversion operation on the added received signal and generates image data for display, and a display part 26 to display the image data for display.

In this embodiment, a procedure from the transmission/reception of ultrasonic waves to the storage of received signals into the receiving buffer 16 is the same as the first embodiment. In this embodiment, the displacement detection of the object to be tested is performed on the received signal. While image data is expressed in 256 gradations of 8 bits in many cases, the received signal is generally expressed by more bits. Besides, according to a structure, an ultrasonic signal can also be expressed as a complex signal after phase detection. Thus, it is possible to detect the displacement by using more information. The detection of displacement can be performed in such a manner that the received signal is regarded as an array of two-dimensional data, and a method such as block matching is used similarly to the case of the image.

In the signal addition part 22, the displacement corrected signal is generated using the detected displacement, and the displacement corrected received signals of plural frames are weight-added to generate an added received signal. In this embodiment, since the coordinate system of the displacement detection and the coordinate system of the displacement corrected received signal are the same, conversion is not required, and an interpolation processing to calculate displacement at an arbitrary position is merely required.

Finally, the filter operation or the scan conversion operation is performed on the added received signals to generate an image for display. The generated image for display is presented to the user by the display part 26.

In this embodiment, since the processing to convert the displacement is unnecessary, and the one image generation part 28 is sufficient, the structure can be made simpler.

### Fourth Embodiment

An ultrasonic diagnostic apparatus 10 according to a fourth embodiment of the invention will be described with reference to FIG. 10. This embodiment is different from the first embodiment in that displacement detection is performed on a received signal and signal addition is performed on image data.

FIG. 10 is a block diagram showing the ultrasonic diagnostic apparatus 10 of this embodiment.

The ultrasonic diagnostic apparatus 10 includes an ultrasonic probe 12 configured to transmit/receive ultrasonic waves, a receiving part 14 which drives the ultrasonic probe 12 to transmit ultrasonic waves, receives the reflected ultrasonic waves, and performs A/D conversion processing to convert them into received signals, a receiving buffer 16 to hold the converted received signals, a displacement detection part 20 to detect displacement between frames of the received signals at each local part of an object to be tested, an image generation part 30 to perform a filter operation or a scan conversion operation on the received signals to generate image data for display, a signal addition part 22 to weight-add image data of plural frames by using the detected displacement information, and a display part 26 to display the added image data for display.

A procedure until a displacement detection processing in this embodiment is the same as the third embodiment, and the displacement detection of an object to be tested is performed on the received signal.

In this embodiment, data to be added in the signal addition part 22 is image data. While the displacement detection processing is performed, the image for display is generated in the image generation part 30. In the image generation part 30, the image data for display is generated from the received signals held in the receiving buffer 16 by the filter operation or the scan conversion operation.

In the signal addition part 22 in this embodiment, first, the displacement information on the received signal detected in the displacement detection part 20 is converted into the displacement in the coordinate system of a frame of an image for display, and the displacement corrected image data is obtained by using the converted displacement. Since the displacement information is detected only at the feature point position on the received signal, a spatial interpolation processing is performed so that displacement at an arbitrary position can be calculated. Thereafter, the interpolated displacement information is converted into that in the coordinate system of the image data for display, and the displacement corrected image is generated by using this. The spatial interpolation processing may be performed in the coordinate system of the image data for display.

Further, in the signal addition part 22, the displacement corrected images of plural frames are weight-added to generate an added image.

Finally, the added image is presented to the use by the display part 26.

By adopting the structure as stated above, the conversion of the displacement information is the conversion from the coordinate system of the received signal to the coordinate system of the image data for display, and since this conversion is the conversion generally required in the generation of the image for display, this conversion function portion can be shared with the image generation part 30, and the structural elements can be decreased.

### MODIFIED EXAMPLE

The invention is not limited to the above embodiments, but can be variously modified within the scope not departing from its gist.

What is claimed is:

1. An ultrasonic diagnostic apparatus which transmits ultrasonic waves to an object to be tested and acquires time-series received signals representing the ultrasonic waves reflected from the object, the ultrasonic diagnostic apparatus comprising:
a first image converter which converts the time-series received signals into time-series images;
a displacement detector which detects information relating to displacement between the time-series images;
a displacement corrector which inversely converts the information relating to the displacement and generates, based on the inversely converted information relating to the displacement, plural displacement corrected signals in which a position shift due to a movement of the object is corrected;
an addition processor which generates an added received signal by weight-adding the time-series received signals and the plural displacement corrected received signals; and
a second image converter which converts the added received signal into an output image.

2. The ultrasonic diagnostic apparatus according to claim 1, further comprising a display which displays the output image and at least one of the time-series images.

3. An ultrasonic diagnostic apparatus which transmits ultrasonic waves to an object to be tested and acquires time-series received signals representing the ultrasonic waves reflected from the object, the ultrasonic diagnostic apparatus comprising:
an image converter which converts the time-series received signals into time-series images;
a displacement detector which detects information relating to displacement between the time-series images;
a displacement corrector which inversely converts the information relating to the displacement and generates, based on the inversely converted information relating to the displacement, plural displacement corrected signals in which a position shift due to a movement of the object is corrected; and
an addition processor which generates an output image by weight-adding the time-series received signals and the plural displacement corrected received signals.

4. The ultrasonic diagnostic apparatus according to claim 1, wherein the information relating to the displacement between the time-series images is displacement of plural feature points on the time-series images.

5. The ultrasonic diagnostic apparatus according to claim 3, wherein the information relating to the displacement between the time-series images is displacement of plural feature points on the time-series images.

6. An ultrasonic diagnostic apparatus which transmits ultrasonic waves to an object to be tested and acquires time-series received signals representing the ultrasonic waves reflected from the object, the ultrasonic diagnostic apparatus comprising:
a displacement detector which detects information relating to displacement between the time-series received signals;
a displacement corrector which generates, based on the information relating to the displacement, displacement corrected received signals in which a position shift due to a movement of the object is corrected;
an addition processor which generates an added received signal by weight-adding the time-series received signals and the plural displacement corrected received signals; and
an image converter which converts the added received signal into an output image.

7. An ultrasonic diagnostic apparatus which transmits ultrasonic waves to an object to be tested and acquires time-series received signals representing the ultrasonic waves reflected from the object, the ultrasonic diagnostic apparatus comprising:
a displacement detector which detects information relating to displacement between the time-series received signals;
a displacement corrector which generates, based on the information relating to the displacement, plural displacement corrected received signals in which a position shift due to a movement of the object is corrected;
an image converter which converts the plural displacement corrected received signals into displacement corrected images; and
an addition processor which converts the time-series received signals into time-series received images and generates an output image by weight-adding the time-series received images and the plural displacement corrected images.

8. The ultrasonic diagnostic apparatus according to claim 1, wherein a weight coefficient of the weighting is a reciprocal of the number n of frames of the images to be added or the received signals to be added.

9. The ultrasonic diagnostic apparatus according to claim 3, wherein a weight coefficient of the weighting is a reciprocal of the number n of frames of the images to be added or the received signals to be added.

10. The ultrasonic diagnostic apparatus according to claim 6, wherein a weight coefficient of the weighting is a reciprocal of the number n of frames of the images to be added or the received signals to be added.

11. The ultrasonic diagnostic apparatus according to claim 7, wherein a weight coefficient of the weighting is a reciprocal of the number n of frames of the images to be added or the received signals to be added.

12. The ultrasonic diagnostic apparatus according to claim 1, wherein a weight coefficient of the weighting is determined according to accuracy of a result of the displacement detection.

13. The ultrasonic diagnostic apparatus according to claim 3, wherein a weight coefficient of the weighting is determined according to accuracy of a result of the displacement detection.

14. The ultrasonic diagnostic apparatus according to claim 6, wherein a weight coefficient of the weighting is determined according to accuracy of a result of the displacement detection.

15. The ultrasonic diagnostic apparatus according to claim 7, wherein a weight coefficient of the weighting is determined according to accuracy of a result of the displacement detection.

16. An image processing method of an ultrasonic diagnostic apparatus which transmits ultrasonic waves to an object to be tested and acquires time-series received signals representing the ultrasonic waves reflected from the object, the image processing method comprising:
converting the time-series received signals into time-series images;
detecting information relating to displacement between the time-series images;
inversely converting the information relating to the displacement and generating, based on the inversely converted information relating to the displacement, plural displacement corrected signals in which a position shift due to a movement of the object is corrected;
generating an added received signal by weight-adding the time-series received signals and the plural displacement corrected received signals; and
converting the added received signal into an output image.

17. An image processing method of an ultrasonic diagnostic apparatus which transmits ultrasonic waves to an object to be tested and acquires time-series received signals representing the ultrasonic waves reflected from the object, the image processing method comprising:
converting the time-series received signals into time-series images;
detecting information relating to displacement between the time-series images;
generating, based on the information relating to the displacement, displacement corrected images in which a position shift due to a movement of the object is corrected; and
generating an output image by weight-adding the time-series received images and the plural displacement corrected images.

18. An image processing method of an ultrasonic diagnostic apparatus which transmits ultrasonic waves to an object to be tested and acquires time-series received signals representing the ultrasonic waves reflected from the object, the image processing method comprising:
detecting information relating to displacement between the time-series received signals;
generating, based on the information relating to the displacement, plural displacement corrected received signals in which a position shift due to a movement of the object is corrected;
generating an added received signal by weight-adding the time-series received signals and the plural displacement corrected received signals; and
converting the added received signal into an image for display.

19. An image processing method of an ultrasonic diagnostic apparatus which transmits ultrasonic waves to an object to be tested and acquires time-series received signals representing the ultrasonic waves reflected from the object, the image processing method comprising:
detecting information relating to displacement between the time-series received signals;
generating, based on the information relating to the displacement, plural displacement corrected received signals in which a position shift due to a movement of the object is corrected;
converting the plural displacement corrected received signals into displacement corrected images respectively; and
generating an output image by converting the time-series received signals into time-series received images and weight-adding the time-series received signals and the plural displacement corrected images.

20. A program product for processing a time-series received signals acquired by scanning an object with ultrasonic waves, the program comprising instructions of:
converting the time-series received signals into time-series images;
detecting information relating to displacement between the time-series images;
inversely converting the information relating to the displacement and to generate, based on the inversely converted information relating to the displacement, plural displacement corrected signals in which a position shift due to a movement of the object is corrected;
generating an added received signal by weight-adding the time-series received signals and the plural displacement corrected received signals; and
converting the added received signal into an outpu image.

21. A program product for processing a time-series received signals acquired by scanning an object with ultrasonic waves, the program comprising instructions of:
converting the time-series received signals into time-series images;
detecting information relating to displacement between the time-series images;
generating, based on the information relating to the displacement, displacement corrected images in which a position shift due to a movement of the object is corrected; and
generating an output image by weight-adding the time-series received images and the plural displacement corrected images.

22. A program product for processing a time-series received signals acquired by scanning an object with ultrasonic waves, the program comprising instructions of:
detecting information relating to displacement between the time-series received signals;
generating, based on the information relating to the displacement, plural displacement corrected received signals in which a position shift due to a movement of the object is corrected;
generating an added received signal by weight-adding the time-series received signals and the plural displacement corrected received signals; and
converting the added received signal into an output image.

23. A program product for processing a time-series received signals acquired by scanning an object with ultrasonic waves, the program comprising instructions of:
detecting information relating to displacement between the time-series received signals;
generating, based on the information relating to the displacement, plural displacement corrected received signals in which a position shift due to a movement of the object is corrected;
converting the plural displacement corrected received signals into displacement corrected images; and
generating an output image by converting the time-series received signals into time-series received images and weight-adding the time-series received images and the plural displacement corrected images.
